(19) United States
(12) Patent Application Publication
(10) Pub. No.: US 2023/0384300 A1
(43) Pub. Date: Nov. 30, 2023

(54) ELECTROCHEMILUMINESCENT NANOPROBE, PREPARATION METHOD THEREOF, ELECTROCHEMILUMINESCENCE DETECTION METHOD FOR NUCLEIC ACID SPECIFIC SITE MODIFICATION, KIT FOR ELECTROCHEMILUMINESCENCE DETECTION METHOD USING ANTIBODY, AND NANOPARTICLE FOR ELECTROCHEMILUMINESCENT NANOPROBE

(71) Applicants: CANON MEDICAL SYSTEMS CORPORATION, Tochigi (JP); Nanjing University, Nanjing (CN)

(72) Inventors: Hao HUANG, Beijing (CN); Qiqi XU, Beijing (CN); Xiaotian WU, Beijing (CN); Huangxian JU, Nanjing (CN); Jie WU, Nanjing (CN)

(73) Assignees: CANON MEDICAL SYSTEMS CORPORATION, Tochigi (JP); Nanjing University, Nanjing (CN)

(21) Appl. No.: 18/325,445

(22) Filed: May 30, 2023

(30) Foreign Application Priority Data
May 27, 2022 (CN) 202210592343.6
May 26, 2023 (JP) 2023-087169

(51) Int. Cl. G01N 33/543; B82Y 15/00; G01N 33/58

(52) U.S. Cl. CPC G01N 33/54326; G01N 33/582; B82Y 15/00

(57) ABSTRACT

A method for preparing an electrochemiluminescent nanoprobe according to an embodiment includes: adding a metal complex ion to an inorganic oxide nanoparticle to provide a metal-doped inorganic oxide nanoparticle; and binding a secondary antibody to the metal-doped inorganic oxide nanoparticle to provide a metal-doped inorganic oxide nanoparticle modified with the secondary antibody. The secondary antibody is configured to identify a specific antibody against nucleic acid-specific site modification.

### CROSS-REFERENCE TO RELATED APPLICATIONS

This application is based upon and claims the benefit of priority from Chinese Patent Application No. 202210592343.6, filed on May 27, 2022; and Japanese Patent Application No. 2023-087169, filed on May 26, 2023, the entire contents of all of which are incorporated herein by reference.

### REFERENCE TO A SEQUENCE LISTING

In accordance with 37 CFR § 1.52(e) (5) and with 37 CFR § 1.831, the specification makes reference to a Sequence Listing submitted electronically as a .xml file named “G81002333A ST26.xml”. The .xml file was generated on May 30, 2023 and is 11,608 bytes in size. The entire contents of the Sequence Listing are hereby incorporated by reference.

### FIELD

Embodiments disclosed in this description and drawings refers to electrochemiluminescent nanoprobes, preparation method thereof, electrochemiluminescence detection method for nucleic acid-specific site modification, kits for electrochemiluminescence detection methods using antibodies, nanoparticles for electrochemiluminescent nanoprobes, and electrochemiluminescent nanoprobes.

### BACKGROUND

Various chemical modifications exist in nucleic acids, e.g., deoxyribonucleic acid (DNA) and ribonucleic acid (RNA), These chemical modifications involve dynamical regulation of gene expression as a regulatory mechanism, provided that the nucleic acid sequence is not altered.

DNA methylation, an apparent modification that occurs at the fifth carbon atom of cytosine and can be stably inherited, and exists widely in animal and plant genomes. DNA methylation is widely involved in various physiological processes during mammalian growth and development, including gene silencing, genomic imprinting, x chromosome inactivation, and disease development.

Since the development of some tumors is accompanied by hypermethylation events of specific genes, and since local hypermethylation events of genes occur earlier than malignant growth of cells, detection of methylation levels of specific genes can be an important basis for tumor prediction and diagnosis in early stages. For example, screening detection of colorectal cancer (CRC) in early stage is performed based on apparent genetic biomarkers, and the Food and Drug Administration (FDA) has already approved the detection of increased specific gene promoter CpG methylation for early screening and adjunctive diagnosis of CRC (see Yvette N Lamb et al., Epi proColon (registered trademark) 2.0 CE: A Blood-Based Screening Test for Colorectal Cancer, Mol Diagn Ther. 2017 April; 21(2): 225-232). Tumor suppressor genes triggering hypermethylation differ depending on types of tumor. For example, in ovarian cancer, the tumor suppressor genes RASSF1A, BRCA1, APC, CDKN2A and the like are hypermethylated. In breast cancer, the tumor suppressor genes PCDHB15, WBSCRF17, IGF1, GYPC and the like are hypermethylated (see Tingting Hong, Selective detections of epigenetic modification in DNA, Wuhan University, 2017, Ph.D dissertation). Detecting methylation levels of these specific tumor suppressor genes not only enhances screening and diagnosis of specific tumors, but also helps the evaluation of the efficacy of definitive treatment for tumors and prognostic observation.

Currently, the standard method for DNA methylation detection is hydrogen sulfite treatment (bisulfite treatment), specifically, treatment of DNA with hydrogen sulfite, e.g., sodium hydrogen sulfite, to convert cytosine (C) in DNA to uracil (U). In contrast, methylated 5-methylcytosine (5-mC) is retained unchanged, whereby the next PCR or sequencing can distinguish between 5-mC and C and therefore detection of methylated DNA is achieved.

However, hydrogen sulfite treatment requires pretreatment of nucleic acids under stringent chemical and temperature conditions, and detection operations for PCR or sequencing are complicated and require specialized technicians and specialized equipment. For these reasons, the detection method is inefficient, time consuming, and expensive. Therefore, there is a need to develop a simple, rapid, and highly sensitive methylated DNA detection method.

Recently, methylated DNA detection methods based on the recognition of methylation sites by specific antibodies have attracted attention. Such detection methods do not require pretreatment of nucleic acids, and enables electrochemical (e.g., Eloy Povedano et al., Amperometric Bioplatforms T₀ Detect Regional DNA Methylation with Single-Base Sensitivity, Anal. Chem, 2020, 92, 5604-12) detection, fluorescent detection and other types of detection.

The recently developed ECL (Electrochemiluminescence) technology has already been widely applied in biological analysis, e.g., detection of tumor protein-labeled substances (e.g., Xiaoming Zhou et al., Synthesis, labeling and bioanalytical applications of a tris(2,2′-bipyridyl)Ruthenium(II)-based electrochemiluminescence probe, Nat Protoc 2014 May; 9(5)). ECL technology is a method that utilizes electrochemical principles to cause an electrochemical reaction on an electrode surface, generating excited states to trigger specific luminescence. ECL is electroluminescence, and therefore does not require an additional excitation light source and is not affected by light fading, light interference and the like, compared to fluorescence. ECL has the advantages in terms of simple equipment, low cost, low background signal, and high detection sensitivity.

However, there is no adequate ECL detection method to detect nucleic acid-specific site modification in conventional techniques.

One of the problems to be solved by embodiments disclosed in this description and in drawings is to provide a simple, sensitive, rapid, and versatile electrochemiluminescence detection method for nucleic acid-specific site modification. Furthermore, this embodiment provides electrochemiluminescence nanoprobes used for the above detection method, a method for their preparation, and a kit for the above detection method. However, the problems to be solved by the embodiments disclosed in this description and in the drawings are not limited to the above problems. Problems corresponding to advantageous effects brought by configurations in the embodiment described below can also be interpreted as other problems.

### BRIEF DESCRIPTION OF THE DRAWINGS

FIG. 1 is a schematic diagram illustrating a detection flow according to one of present embodiment(s) of an electrochemiluminescence detection method.

FIG. 2 is a schematic diagram illustrating a method of preparing a reaction reagent 1 used in the electrochemiluminescence detection method according to the present embodiment.

FIG. 3 is a schematic diagram illustrating a method of preparing a reaction reagent 3 used in the electrochemiluminescence detection method according to the present embodiment.

FIG. 4A is a picture representing a result of characterizing ECL nanoprobes prepared in Example 1.

FIG. 4B is a diagram representing a result of characterizing ECL nanoprobes prepared in Example 1.

FIG. 5A is a diagram representing a result of characterizing ECL nanoprobes prepared in Example 1.

FIG. 5B is a diagram representing a result of characterizing ECL nanoprobes prepared in Example 1.

FIG. 5C is a diagram representing a result of characterizing ECL nanoprobes prepared in Example 1.

FIG. 6 is a diagram representing a relationship between a storage time for the ECL nanoprobes prepared in Example 1 and ECL intensity.

FIG. 7A is a diagram representing a result of measurement for the ECL sensor system prepared in Example 3.

FIG. 7B is a diagram representing a result of measurement for the ECL sensor system prepared in Example 3.

FIG. 8A is a diagram representing ECL response statuses under different ECL detection conditions of Ru@SiO₂ prepared in Example 3.

FIG. 8B is a diagram representing ECL response statuses under different ECL detection conditions of Ru@SiOprepared in Example 3.

FIG. 9A is a diagram representing ECL response statuses under different detection conditions of the ECL sensor system prepared in Example 3.

FIG. 9B is a diagram representing ECL response statuses under different detection conditions of the ECL sensor system prepared in Example 3.

FIG. 9C is a diagram representing ECL response statuses under different detection conditions of the ECL sensor system prepared in Example 3.

FIG. 9D is a diagram representing ECL response statuses under different detection conditions of the ECL sensor system prepared in Example 3.

FIG. 10 is a picture for investigation of fluorescence representing principle of detection of target nucleic acids by the ECL sensor system prepared in Example 3.

FIG. 11A is a diagram representing ECL response statuses at different concentrations of methylated DNA in the ECL sensor system prepared in Example 3.

FIG. 11B is a diagram representing ECL response statuses at different concentrations of methylated DNA in the ECL sensor system prepared in Example 3.

FIG. 12 is a diagram representing a result of evaluating selectivity of the ECL detection method according to the present embodiment.

FIG. 13A is a diagram representing a result of evaluating stability of the ECL detection method according to the present embodiment.

FIG. 13B is a diagram representing a result of evaluating stability of the ECL detection method according to the present embodiment.

### DETAILED DESCRIPTION

The following is a detailed description of a specific embodiment of the present embodiment with reference to drawings. The following description of the embodiment is intended only to explain the inventive concept of this embodiment and is not intended to limit this embodiment.

This embodiment provides a method for electrochemiluminescence detection of nucleic acid-specific site modification with use of a nanoprobe, an electrochemiluminescence nanoprobe used for the above detection method, a method of preparing the same, and a kit used for the above detection method.

One aspect of the present embodiment refers to a method for electrochemiluminescence detection of nucleic acid specific site modification with use of the nanoprobe. The method refers to an electrochemiluminescence detection method (also referred to as ECL detection method, hereinafter) for the nucleic acid specific site and includes: a step 1 (first step) of mixing a sample to be detected with a magnetic bead(s) that serves as a reaction reagent 1 (first reaction reagent) and is modified with a capture nucleic acid, in order to identify and capture the modification of the target nucleic acid(s) in the sample; a step 2 (second step) of capturing and labeling the specific site modification of the target nucleic acid with use of the specific antibody that serves as a reaction reagent 2 (second reaction reagent) and is used for the nucleic acid specific site modification; a step 3 (third step) of performing detection signal labeling to the composite that is obtained at the step 2 and contains the magnetic bead capture nucleic acid-target nucleic acid-specific antibody with use of the electrochemiluminescence nanoprobe prepared by the above preparation method as a reaction reagent 3 (third reaction reagent); and a step 4 (fourth step) of placing the composite that is obtained at the step 3 and contains the magnetic bead capture nucleic acid-target nucleic acid-specific antibody-nanoprobe, on the electrode surface, performing the electrochemiluminescence detection after adding a co-reactive agent, and then performing qualitative and quantitative analysis for the modification of the target nucleic acid based on the presence/absence and intensity of the electrochemiluminescence detection signal.

In this embodiment that involves detecting the nucleic acid-specific site modification by electrochemiluminescence method, the nucleic acid is DNA or RNA, for example. The aforementioned nucleic acid-specific site modification is not particularly limited, and may be methylation modification (e.g., methylation of 5-methylcytosine also referred to as 5mC hereinafter) of DNA, hydroxymethylation modification (e.g., hydroxymethylation of 5-methylcytosine also referred to as 5hmC hereinafter), pseudouridylation of RNA (e.g., methylation of the sixth N of adenine, also referred to as m6A hereafter), or the like. Hydroxymethylation modification is also called methylolation modification.

Electrochemiluminescence systems are categorized mainly into two types depending on the luminescent reagents: (1) metal complex electrochemiluminescence systems and (2) organic compound electrochemiluminescence systems containing fused ring aromatic hydrocarbons and hydrazides. Electrochemiluminescent metal complexes typically used include ion complexes of metals such as Ru, Os, Re, Ir, Cr, Pd, Al, Cd, Pt, Mo, Tb, Eu or the like. Among them, metal complexes of Ru, Ir, Os or Re have attracted attention because of their good electrochemiluminescence properties.

Among Ru metal complexes, tris(bipyridine)ruthenium(II) complex ion (Ru(bpy)₃²⁺ is widely applied due to its high water solubility, stable chemical performance, reversible redox, high luminescence efficiency, wide pH range for application, electrochemical regeneration, and long excited state lifetime. Here, Ru(bpy)₃²⁺ reacts with tripropylamine (TPrA), the co-reactive agent, to enable ECL detection under low potential conditions. The reaction equation between Ru(bpy)₃²⁺ and tripropylamine TPrA is as follows.

Tripopylamine→Triporpylamine·+e− (1)

Ru(bpy)₃²⁺→Ru(bpy)₃³⁺+e− (2)

Ru(bpy)₃³⁺+Tripropylamine·→[Ru(bpy)₃²⁺]*+products (3)

[Ru(bpy)₃²⁺]*→Ru(bpy)₃²⁺+hv (4)

The electrochemiluminescence detection method in the present embodiment is preferably performed with ruthenium (II) complex ion systems, but can be performed with other metal complex ion systems, such as iridium (III) complex ion system.

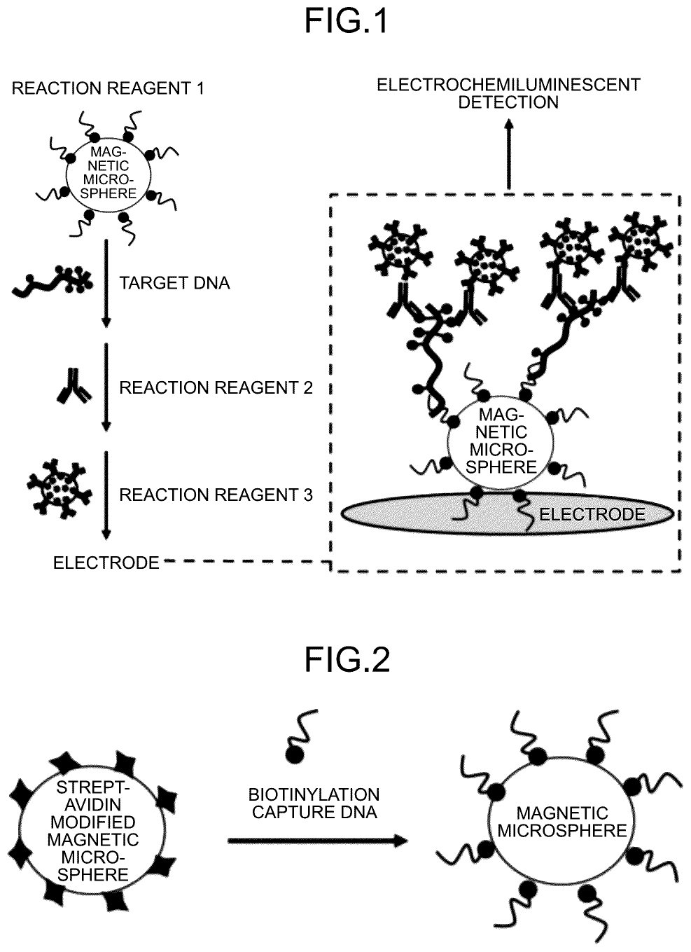

FIG. 1 is a schematic diagram of the detection flow of the electrochemiluminescence detection method in the present embodiment. As illustrated in FIG. 1, reaction reagent 1, reaction reagent 2, and reaction reagent 3 are used in the ECL detection method in the present embodiment.

Specifically, the reagent 1 is the magnetic bead modified with the capture nucleic acid. The capture nucleic acid is designed for the target nucleic acid and has a nucleic acid sequence complementary to the target nucleic acid, allowing for stable and specific hybridization of the two nucleic acids. The capture nucleic acid can be synthesized by a method common in the relevant field, or may be a commercially available product to be used directly.

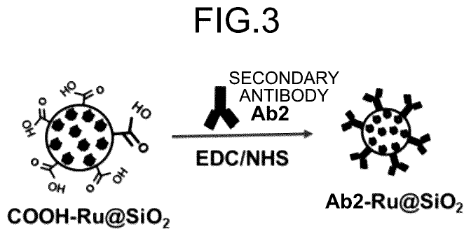

FIG. 2 is a schematic diagram representing the method of preparing the reaction reagent 1 to be used in the electrochemiluminescence detection method in this embodiment. As illustrated in FIG. 2, the capture nucleic acid sequence may be modified with biotin at the terminal, or streptavidin may be bound to the magnetic bead. The capture nucleic acid may be modified to the magnetic bead by the biotin-streptavidin reaction. The streptavidin-bound magnetic bead (also referred to as S-MBs, hereinafter) can be synthesized by general methods in the relevant field, or may be a commercially available product to be used directly. The above biotin-streptavidin binding method is the most conventional method in the relevant field, the binding method between the capture nucleic acid and the magnetic bead is not limited to this method, and can be any method that can bind the capture nucleic acid to the magnetic bead.

The reagent 2 is a specific antibody (also referred to as primary antibody, hereinafter) against the specific site modification of the target nucleic acid. The relevant specific antibody can be prepared by a general preparation method for antibodies in the relevant field depending on the specific site modification, or may be a commercially available product to be used directly. The primary antibody is an antibody against methylated cytosine (5mC) or hydroxymethyl cytosine (5hmC) of DNA, for example.

The reaction reagent 3 is an electrochemiluminescent nanoprobe (also referred to as ECL nanoprobe, hereinafter) prepared in this embodiment, i.e., a metal-doped inorganic oxide nanoparticle modified with a secondary antibody.

One embodiment of this embodiment relates to a method for preparing the electrochemiluminescence nanoprobe, and refers to a method for preparing the electrochemiluminescence nanoprobe (also referred to as ECL nanoprobe, hereinafter) including a step of adding a metal complex ion to an inorganic oxide nanoparticle to obtain a metal-doped inorganic oxide nanoparticle, and a step of binding a secondary antibody to the metal-doped inorganic oxide nanoparticle to obtain metal-doped inorganic oxide nanoparticle modified with the secondary antibody so as to allow the secondary antibody to identify the specific antibody against the nucleic acid-specific site modification. The secondary antibody is configured to identify a specific antibody against nucleic acid-specific site modification.

The nanomaterial is used as a carrier in the preparation of the ECL nanoprobes in this embodiment. The nanomaterial has a huge surface area or porous structure, and therefore can be used for ultrasensitive detection by loading a large amount of luminescent material. In terms of high stability, low cost, and ease of modification, an inorganic oxide nanoparticle is preferred for the nanomaterial used in this embodiment. Specific examples of the inorganic oxide nanoparticles include silicon dioxide nanoparticles, titanium dioxide nanoparticles, zinc oxide nanoparticles, iron oxide nanoparticles, and nanoparticles having surfaces coated with silicon dioxide, titanium dioxide, zinc oxide, iron oxide or the like. Among them, the silicon dioxide nanoparticles are more preferred.

The method of preparing the ECL nanoprobe in this embodiment includes the step of adding the metal complex ion to the inorganic oxide nanoparticle. The addition method can be a conventional addition method in the relevant field, such as electrochemical method, sol-gel method, ion-exchange method, or hydrolytic precipitation method. The method is not particularly limited to the above methods, and may be any method capable of adding a metal complex ion to an inorganic oxide nanoparticle. In other words, the nanoparticle for an electrochemiluminescent nanoprobe comprise an inorganic oxide doped with a metal complex ion.

This method of preparing the ECL nanoprobe in this embodiment further includes a step of binding the secondary antibody to the metal-doped inorganic oxide nanoparticle to obtain the metal-doped inorganic oxide nanoparticle modified with the secondary antibody. Here, the method of binding the secondary antibody to the metal-doped inorganic oxide nanoparticle is preferably by covalent bonding, but may be another method, such as electrostatic adsorption. Specific examples of methods of forming covalent bonds include carboxy-amino bonds and aldehyde group-amino bonds. There is no particular limitation on the reaction to form the covalent bonds, but the covalent bonding reaction is preferably performed at room temperature with rapid reaction and high bonding efficiency. In other words, the electrochemiluminescent nanoprobe comprise an inorganic oxide nanoparticle doped with a metal complex ion and a secondary antibody bound to the inorganic oxide nanoparticle.

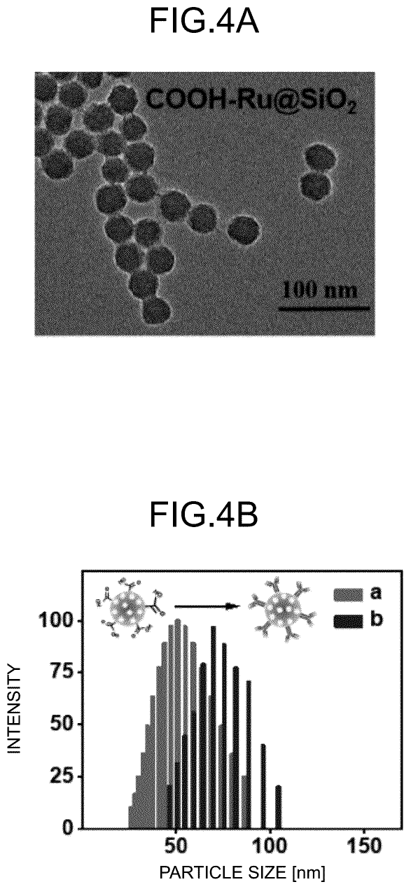

FIG. 3 is a schematic diagram representing the method of preparing the reaction reagent 3 to be used in the electrochemiluminescence detection method in this embodiment. In FIG. 3, the tris(bipyridine)ruthenium(II) complex ion (Ru(bpy)₃²⁺) is used as an example. In FIG. 3, the EDC/NHS system (1-(3-dimethylaminopropyl)-3-ethylcarbodiimide hydrochloride)/N-hydroxysuccinimide system) is used to bind the secondary antibody (also referred to as Ab2, hereafter) to the metal-doped inorganic oxide nanoparticles.

Specifically, Ru(bpy)₃²⁺ is added to silicon dioxide nanoparticles to provide ruthenium-doped silicon dioxide nanoparticles (also referred to as Ru@SiO₂, hereinafter). Next, Ru@SiO₂ is carboxylated to form carboxylated ruthenium-doped silicon dioxide nanoparticles (also referred to as COOH-Ru@SiO₂, hereinafter). Next, the secondary antibody is bound to COOH-Ru@SiO₂ using the EDC/NHS system to form secondary antibody-modified ruthenium-doped silicon dioxide nanoparticles (also referred to as Ab2-Ru@SiO₂, hereinafter).

The ECL nanoprobe in this embodiment can bind a large number of metal complex ions to one nanobead, and can significantly amplify the electrochemical signal, and therefore can greatly increase the sensitivity of ECL detection.

As well, In the ECL nanoprobe according to this embodiment, a plurality of secondary antibody molecules can be bound to one nanobead, thereby improving the binding efficiency between the nanoprobe and the primary antibody.

This achieves great improvement of the sensitivity of ECL detection by using the ECL nanoprobe in this embodiment, thereby achieving detection for the nucleic acid-specific site modification up to fM level.

The secondary antibody in this embodiment identifies the primary antibodies against the nucleic acid-specific site modification. The relevant secondary antibody is preferably designed not for the specific portion of binding to the primary antibody and the nucleic acid-specific site modification, but for generic portion of the primary antibody. In other words, the secondary antibody in this embodiment is preferably a protein that identifies the generic portion of the primary antibody. This allows this embodiment to provide the generic ECL nanoprobe that can be used for various ECL detection methods with antibodies. The aforementioned secondary antibody is not particularly limited but can be prepared by general antibody preparation methods in the relevant field, or a commercially available product to be used directly. The generic portion is also referred to as a stationary region.

Thereby, one embodiment of this embodiment further provides ECL nanoprobes prepared by the method of preparation of the ECL nanoprobe in this embodiment described above. Preferably, the ECL nanoprobe in this embodiment is a generic ECL nanoprobe used in the ECL detection method using antibody.

The ECL nanoprobe prepared in this embodiment has excellent storage stability and can be stored for 10 days or more under storage conditions at room temperature, for example. Therefore, the ECL nanoprobe in this embodiment can be used as it is after the preparation, but it can be used also when needed after the storage for a certain amount of time.

Thereby, another embodiment in this embodiment provides a kit used for the method of electrochemiluminescence detection with antibodies. The kit includes the ECL nanoprobe in this embodiment described above. In other words, the kit for a method of electrochemiluminescence detection with an antibody, the kit comprising an electro-chemiluminescent nanoprobe prepared by an electro-chemiluminescent nanoprobe preparation method including: adding a metal complex ion to an inorganic oxide nanoparticle to provide a metal-doped inorganic oxide nanoparticle; and binding a secondary antibody to the metal-doped inorganic oxide nanoparticle to provide a metal-doped inorganic oxide nanoparticle modified with the secondary antibody, the secondary antibody being configured to identify a specific antibody against nucleic acid-specific site modification.

In addition to the ECL nanoprobe, the kit can include various reagents required for the intended use, such as the reaction reagents 1 and 2 and instructions for use.

The ECL detection method in this embodiment may specifically include the following steps 1 to 4, as illustrated in FIG. 1.

Step 1: The reagent 1 (e.g., S-MBs/B-Cap) is mixed with the sample (e.g., target DNA) to be detected in order to identify and capture the target nucleic acids in the sample. The capture nucleic acid (e.g., capture DNA) bound to the B-Cap is designed to be sequence complementary to the target DNA sequence, and therefore can bind to the target nucleic acid by hybridization after the sample to be detected is added. This allows the target DNA to be captured by the magnetic beads.

After the S-MBs/B-Cap identifies the target DNA, the reaction system may be blocked to prevent nonspecific adsorption of immunoreagents. The blocking can be performed by employing heterologous proteins or detergents, such as Tween-20, BSA, animal serum, nonfat milk powder, and is preferably BSA.

Step 2: The reaction reagent 2 (e.g., Ab-5mC) is added to capture and label the target DNA. The reaction reagent 2, the primary antibody, is designed according to specific antibodies against nucleic acid-specific site modification and can identify the specific site modification in the target nucleic acids. This enables capturing and labeling the specific site modification of the target nucleic acid, forming a magnetic bead-capture DNA-target DNA-Ab-5mC composite, for example.

In terms of achieving high ECL signal intensity, it is preferred to optimize the primary antibody concentration and incubation time after addition of the primary antibody. The primary antibody concentration may be 1.0 μg/mL or more, for example, specifically 2.0 μg/mL or more, 2.5 μg/mL or more, 5 μg/mL or more, 10 μg/mL or more, 20 μg/mL or more, or the like, and is preferably 2.5 μg/mL to 20 μg/mL. The incubation time for the primary antibody may be 5 min to 80 min, for example, specifically 5 min, 10 min, 20 min, 30 min, 40 min, 50 min, 60 min, 70 min, 80 min, and is preferably 20 min to 40 min.

Step 3: The reaction reagent 3 (e.g., Ab2-Ru@SiO₂) is added to perform detection signal labeling to the target DNA. The secondary antibody bound to the reaction reagent 3 can bind specifically to the composite formed at the step 2, thereby enabling performing the detection signal labeling to the composite to form a magnetic bead-capture DNA—target DNA-Ab-5mC-Ab2-Ru@SiO₂ composite.

In terms of achieving high ECL signal intensity, it is preferred to optimize the ECL nanoprobe concentration and incubation time after the addition of the ECL nanoprobe. The ECL nanoprobe concentration may be 1 μg/mL or more, for example, specifically 3 μg/mL or more, 4 μg/mL or more, 6 μg/mL or more, 9 μg/mL or more, 12 μg/mL or more, 15 μg/mL or more, 18 μg/mL or more, and is preferably 4 μg/mL to 15 μg/mL. The incubation time of the ECL nanoprobe may be 5 min to 80 min, for example, specifically 5 min, 10 min, 20 min, 30 min, min, 50 min, 60 min, 70 min, 80 min, and is preferably 20 min to 40 min.

Step 4: The composite obtained at the step 3 is placed on the electrode surface for electrochemiluminescence detection. Here, it is preferred to optimize the pH value of the ECL detection electrolyte and the concentration of the co-reactive agent (e.g., TPrA) in terms of achieving high ECL signal intensity. The pH value of the ECL detection electrolyte may be 5.0 to 10.0, for example, specifically 6.0, 6.5, 7.0, 7.5, 8.0, 8.5, 9.0, 10.0, and is preferably 6.5 or more and more preferably 7.4. The concentration of the co-reactant may be 5.0 to 160.0 mM, for example, specifically mM or more, 20.0 mM or more, 40.0 mM or more, 80.0 mM or more, and is preferably 20.0 mM to 80.0 mM and more preferably 40.0 mM to 80.0 mM.

As illustrated on the right side of FIG. 1, the composite obtained at the step 3 is formed of the magnetic beads, the capture DNA, the target DNA, the primary antibody, and the ECL nanoprobe. When placed on the electrode surface, the composite can generate an electrochemiluminescence signal.

Qualitative and quantitative analysis can be performed for the nucleic acid-specific site modification of the sample detected based on the presence/absence and intensity of the ECL signal. For example, if ECL signal is not detected in the reaction system, it is determined that the detected sample does not contain the nucleic acid-specific site modification and the above composite is not formed to generate electrochemiluminescence signals. In addition, as verified in this embodiment, a linear relationship is achieved between the intensity of electrochemiluminescence and logarithm of the methylated DNA concentration, allowing for quantitative analysis of methylated DNA based on the intensity of electrochemiluminescence.

In this embodiment, the electrochemiluminescence signal can be detected using an electrochemical chemical analysis system customarily used in the relevant field. The electrode used may be a typical electrode, for example, one of glass carbon electrodes, indium tin oxide (ITO) electrodes and screen printed electrodes. In this embodiment, it is preferred to use a standard three-electrode system in which a platinum wire electrode, a Ag/AgCl reference electrode, and a glass carbon working electrode (GCE) are arranged.

This ECL detection method is not limited to the above step. The above step is initiated by binding the primary antibody to the hybrid product of the target nucleic acid-capture nucleic acid, followed by adding the ECL nanoprobe according to this embodiment to form the primary antibody-secondary antibody composite. However, it is possible to form the primary antibody-secondary antibody composite by binding the primary antibody to the metal-doped inorganic oxide nanoparticle modified with the secondary antibody and then bind this antibody composite to a hybrid product of the target nucleic acid-capture probe. This enables forming the composite that contains the magnetic bead-captured nucleic acid-target nucleic acid-primary antibody-ECL nanoprobe, as well. Furthermore, in this embodiment, the primary antibody can be directly modified on the metal-doped inorganic oxide nanoparticles modified with the secondary antibody to form a product to be entirely used as an ECL nanoprobe for detection.

### EXAMPLES

Hereinafter, this embodiment is specifically explained with reference to Examples. However, the Examples are merely described as examples, and this embodiment is not limited to the Examples.

Note that all reagents used in the Examples are at the analytical purity level.

The meanings of abbreviations used are as follows:
Ru(bpy)₃²⁺: Tris(2,2′-bipyridine)dichlororuthenium(II) hexahydrate
TEOS: Tetraethyl orthosilicate
NH₄OH: Ammonia water
Ru@SiO₂: Ruthenium-doped silicon dioxide nanoparticles
Ab2-Ru@SiO₂: Ruthenium-doped silicon dioxide nanoparticles modified with secondary antibody
CTES: Carboxyethylsilanetriol Na salt
COOH-Ru@SiO₂: Carboxylated ruthenium-doped silicon dioxide nanoparticle
DI water: Deionized water
EDC: N-(3-dimethylaminopropyl)-N′-ethylcarbodiimide hydrochloride
NHS: N-hydroxysuccinimide
MES: 2-(N-morpholine) ethanol sulfonic acid
PBS: phosphate buffer solution
BSA: Bovine serum protein
S-MBs: Streptavidin magnetic beads
B-Cap: Biotin-capture DNA
T: DNA sample
Ab-5mC: Anti 5-methylcytosine antibody

### Example 1: Preparation of Reagent 3 (ECL Nanoprobe)

### (1) Preparation of Ru@SiO₂

1. 7.5 mL of cyclohexane (Aladdin (Shanghai, China)) was mixed with 1.8 mL of n-hexanol (TCI chemicals (Shanghai, China)) and 1.77 mL of Triton X-100 (Sigma (Shanghai, China)) for 15 minutes.
2. 340 μL of Ru(bpy)3 2 +: (Sigma (Shanghai, China)) (40 mM) aqueous solution was added and then mixed for 30 minutes.
3. 100 μL of TEOS (Sigma (Shanghai, China)) was added and then mixed for 30 minutes.
4. 60 μL of NH₄OH (Macklin (Shanghai, China)) was added and then mixed for 24 hours under light-shielded conditions to provide Ru@SiO₂.

### (2) Preparation of Ab2-Ru@SiO₂

1. 50 μL of CTES (J&K Scientific (Shanghai, China)) was added and mixed for 7 hours under light-shielded conditions to provide COOH-Ru@SiO₂.
2. The product was washed sequentially with 15 mL of acetone, ethanol and DI water, and subjected to centrifugal separation at 10000 rpm for 10 minutes, and dispersed in DI water.
3. 10 μL of 50 mg/mL COOH-Ru@SiO₂ was taken and activated with 500 μL of EDC/NHS (50 mg/mL each in 25 mM MES (PH 5.5), both EDC and NHS were purchased from J&K Scientific (Shanghai, China)) for 35 minutes. Then, the product was washed with 10 mM PBS (pH 7.4) for 5 minutes at 12000 rpm and dispersed in 1 mL of 10 mM PBS.
4. 20 μL of 2 mg/mL Ab2 (rabbit anti-mouse IgG antibody (product number: ab6709), purchased from Abcam (Shanghai, China)) was added. Then, the resulting product was shaken at rpm for 4 hours.
5. The product was washed at 12000 rpm for 5 minutes with 25 mM MES buffer and 10 mM PBS to provide Ab2-Ru@SiO₂.
6. Finally, the resulting Ab2-Ru@SiO₂ was stored in 0.1% BSA (KeyGEN BioTECH (Jiangsu, China)/5 mM PBS buffer at 4° C. until time of use.

### Example 2: Characterization of ECL Nanoprobe

### A: Confirmation of COOH-Ru@SiO₂ Formation by TEM

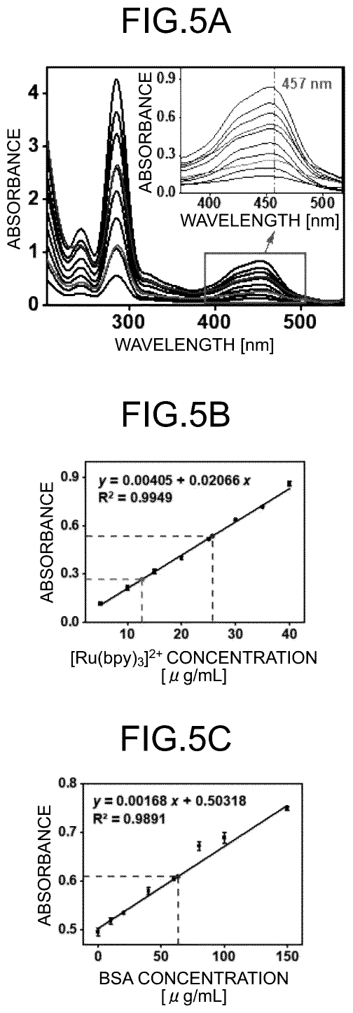

COOH-Ru@SiO₂ nanoparticles obtained in Example 1 were photographed with a JEM-2100 transmission electron microscope (JEOL). The resulting transmission electron micrograph (TEM) is illustrated in FIG. 4A. FIGS. 4A and 4B represent results of characterizing the ECL nanoprobes prepared in Example 1. FIG. 4A is a TEM photograph of COOH-Ru@SiO₂ obtained in Example 1. FIG. 4B represents grain size distribution maps of COOH-Ru@SiO₂ (a) and Ab2-Ru@SiO₂ (b) obtained by DLS.

As recognized from FIG. 4A, the resulting COOH-Ru@SiO₂ nanoparticles are homogeneously dispersed, uniform in size, and have spherical shapes each having a diameter of approximately 30 nm.

### B: Confirmation of Ab2-Ru@SiO₂ Formation by Dynamic Light Scattering (DLS) Method

The particle size distributions of COOH-Ru@SiO₂ and Ab2-Ru@SiO₂ were measured by dynamic light scattering (DLS) method using a 90 Plus/BI-MAS instrument (Brookhaven, USA). FIG. 4B illustrates the results.

As recognized from FIG. 4B, the hydrated particles of COOH-Ru@SiO₂ (a) were distributed at approximately 50 nm in size, with a narrow particle size distribution, uniform size, and monodispersity. The hydrated particle size of Ab2-Ru@SiO₂ (b) is approximately 70 nm, slightly larger than that of COOH-Ru@SiO₂, demonstrating coupling between Ab2 and Ru@SiO₂ was succeeded while achieving high dispersibility without aggregation phenomenon.

### C: Confirmation of Number of Ruthenium Molecules Bound to Ab2-Ru@SiO₂ and Number of Bound Antibodies

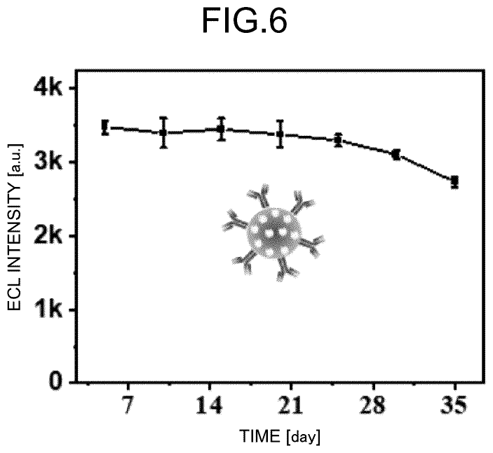

T₀ obtain the number of ruthenium molecules bound to a single Ru@SiO₂, quantitative analysis of UV-visible light spectrum (UV) was performed using the Nanodrop-2000C UV-Vis spectrophotometer (Thermon, USA) for the Ab2-Ru@SiO₂ obtained in Example 1. FIG. 5A illustrates the result. FIGS. 5A, 5B and 5C represent the result of characterizing the ECL nanoprobes prepared in Example 1. FIG. 5A represents the UV-visible spectrum of Ab2-Ru@SiO₂ nanoprobe, FIG. 5B represents the concentration calibration curve of [Rubyp₃²⁺] at 457 nm, and FIG. 5C represents the concentration calibration curve of BSA standard at 620 nm.

As illustrated in FIG. 5A, the absorbance A at 457 nm of 100 μg/mL of Ru@SiO₂ is 0.26. The concentration of [Ru(bpy)_{3]}²⁺ in 100 μg/mL of Ru@SiO₂ is determined to be 12.53 μg/mL based on the [Ru(bpy)_{3]}²⁺ concentration calibration curve in FIG. 5B. With this determination, the number of [Ru(bpy)_{3]}²⁺ molecules in 1 mL of Ru@SiO₂ is estimated to be N_{[Ru(bpy)3]2+}=NA·n=NA·m_{Ru}/M_{Ru}=1.01×10¹⁶. Furthermore, the number of [Ru(bpY)_{3]}²⁺ molecules in a single Ru@SiO₂ is N_{[Rubpy3]2+}/N_{Ru@SiO2}=3.1×10⁴.

The absorbance of Ab2 in Ab2-Ru@SiO₂ obtained in Example 1 was measured at 620 nm by the Bradford method protein quantification kit (Sangon Biotech (Shanghai, China)) for plate reader. SiO₂, the absorbance A620 of Ab2 in Ab2-Ru@SiO₂ is 0.63.

Based on the BSA protein calibration curve in FIG. 5C, the concentration of Ab2 in Ab2-Ru@SiO₂ was determined to be 73.11 μg/mL, yielding the molar mass MAb2 of IgG-Ab2=150 KD=1.5×10⁵ g/mol as well as NAb2=NA·n=NA·mAb2/MAb2=2.94×10¹⁴ for the number of Ab2 in 1 mL of 73.11 μg/mL Ab2.

Based on the number of Ru@SiO₂ in 2 mg of Ru@SiO₂, the number N₂ of Ru@SiO₂ in 1 mL of 1 mg/mL Ru@SiO₂ is determined to be 3.22×10¹³. This enables determining the number of antibodies bound to a single Ru@SiO₂ to be NAb2/N₂=2.94210¹⁴/3.22×10¹³≈9.

### D: Storage Stability of Ab2-Ru@SiO₂

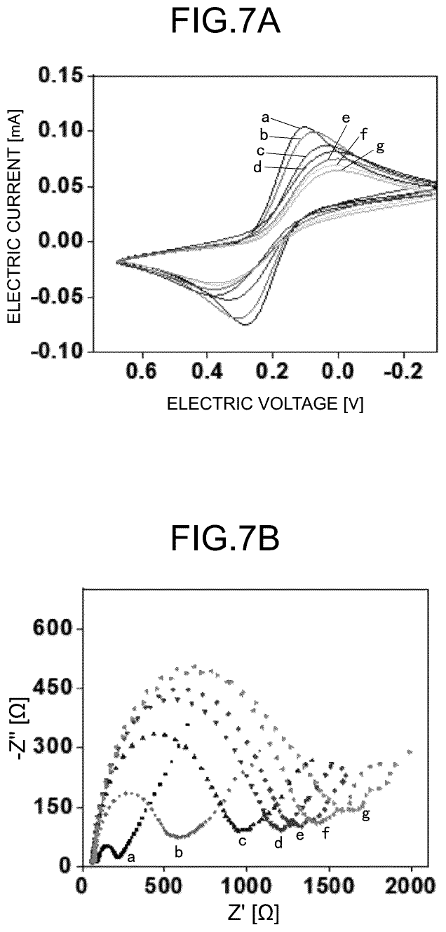

The Ab2-Ru@SiO₂ obtained in Example 1 was stored in a 1% BSA-containing aqueous solution at 4° C. while shaded from light. ECL signal was measured for Ab2-Ru@SiO₂ every 5 days. FIG. 6 illustrates the results. FIG. 6 represents the relationship between ECL intensity and storage time for the ECL nanoprobes prepared in Example 1.

In Examples, all ECLs were measured in an electrolytic cell of MPI-A multifunctional electrochemiluminescence analysis system (Xi'an Remex, China), standard three-electrode system. A platinum wire electrode, a Ag/AgCl reference electrode, and a glass carbon working electrode (GCE) with a diameter of 5 mM were placed in the standard three-electrode system described above.

The ECL response was recorded in ECL detection electrolyte (0.1 M PBS, 0.1 M KNO₃,40 mM TPrA, PH 7.4) using the three-electrode system. With use of an electrochemical cyclic voltammetry (CV) (experiments were performed on a CHI 630 D electrochemical workstation (Shanghai Shenhua, China), potential scanning was performed continuously from 0 to +1.25 V at a scanning rate of 100 mV/s. A photomultiplier tube (PMT) voltage was set at 600 V. All ECL measurements were performed at room temperature. The same applies to measurements described below.

As recognized from FIG. 6, the Ab2-Ru@SiO₂ probe was verified to have excellent storage stability in a 1% BSA-containing aqueous solution in consideration of little change in the ECL intensity for 20 days.

### Example 3: ECL Detection Method

DNA sequences used in this Example were synthesized and purified by Shanghai Sangon Biotech. The sequences used are specifically listed in Table 1.

TABLE 1

| | |
|---|---|
| B-Cap | Biotin-CTCCTTCGTCCCCTCCTCACACCCCACC |
| 0×5mC-RASSF1A | GCTTTGCGGTCGCCGTCGTTGTGGCCGTCCGGGGG |
| (T₀) | GGGTGTGAGGAGGGGACGAAGGAG |
| 7×5mC-RASSF1A | GCTTTGC (M) GGTC (M) GCC (M) GTC (M) |
| (T_{5mc}) | GTTGTGGCC (M) GTCC (M) GGGGTGGGGTGTG |
| Cy5-Pro | AGGAGGGGAC (M) GAAGGAGACAACGACGGCGAC |
| | CGCAAAGC-Cy5 |
| 7×5mC-PCDHGB7 | AGCTGC (M) GC (M) GCAGAGGC (M) GCC |
| (T_{P}) | (M) GGGCC (M) GGCCC (M) GC (M) GGC |
| | AGGTACTATTTCCTTTGCTGCTGCT |

### (1) DNA Sample Preparation

A DNA sample (T) containing the following sequences were prepared.
Negative control sequence: 0×5mC-RASSF1A (T₀) (SEQ ID NO: 2)
Target DNA sequence: 7×5mC-RASSF1A (T_{5mC}) (SEQ ID NO: 3)
Non-target DNA sequence: 7×5mC-PCDHGB7 (Tp) (SEQ ID NO: 5)

### (2) Preparation of Reaction Reagent 1 (S-MBs/B-Cap):

1. 2 μL S-MBs (BioMag (Jiangsu, Wuxi)) was washed with TE buffer (B&W buffer containing 2 M NaCl, pH 7.5).
2. It was mixed with 25 μL of 0.1 μM B-Cap (in pH 7.5 TE buffer) and incubated for 15 minutes at 37° C.
3. It was washed with 10 mM PBS to wash off excess B-Caps, to provide S-MBs/B-Caps.

### (3) Identification to Target DNA

1. 50 μL of T (in 10 mM PBS) was added and incubated at 37° C. for 30 minutes,
2. Next, it was washed with 10 mM PBS to remove unreacted T.

### (4) Identification by Ab-5mC to 5mC

1. 50 μL of 2% BSA (in 10 mM PBS) was added and incubated at 37° C. for 30 minutes.
2. 50 μL of Ab-5mC (product number: ab10805, Abcam (Shanghai, China), 2.5 μg/mL) was added and incubated at 37° C. for 20 minutes.
3. Then, it was washed with 10 mM PBS to wash off unreacted Ab-5mC.
   (5) Labeling with ECL Nanoprobe
1. 50 μL of Ab2-Ru@SiO₂ (6 μg/mL) was added and incubated at 37° C. for 20 minutes.
2. Then, it was washed with 10 mM PBS and dispersed in 65 μL 10 mM PBS.

### (6) ECL Detection

1. The solution of the above S-MBs/B-Cap-T-Ab-5mC-Ab2-Ru@SiO₂ composite (hereafter referred to as composite) was dropped onto the GCE electrode. The GCE electrode was dried, and then immersed in ECL electrolyte solution (0.1 M PBS, 0.1 M KNO₃, 40 mM TPrA, pH 7.4).
2. Potential scanning was performed at 0 to 1.25 V and ECL was collected with PMT600.

### Example 4: Characterization of the ECL Sensor System

The GCE electrode detection system including the above composite obtained in Example 3 is also referred to as ECL sensor system in this embodiment.

(1) Measurement of ECL Sensor System in this Embodiment

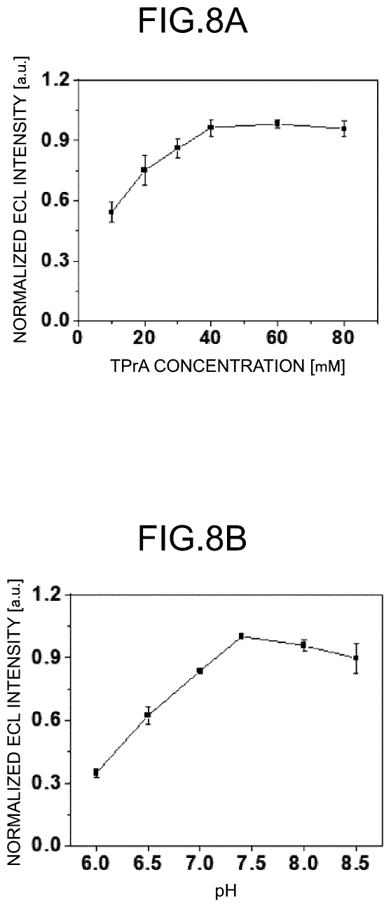

Electrochemical cyclic voltammetry experiment (CV) and electrochemical impedance spectroscopy (EIS) tests were performed on the results obtained at each step for the ECL sensor system prepared in Example 3. Herein, CV was performed on a CHI630D electrochemical workstation (Shanghai Chenhua, China) and EIS tests were performed on a DH7000 (Jiangsu Donghua, China). FIG. 7 illustrates the results. FIGS. 7A and 7B represent the measurement results for the ECL sensor system prepared in Example 3. FIG. 7A is a cyclic voltammetry (CV) diagram demonstrating a stepwise modification of the ECL sensor system. FIG. 7B is an electrochemical impedance spectroscopy (EIS) diagram demonstrating a stepwise modification of the ECL sensor system.

In FIG. 7, a represents the bare GCE electrode, b represents S-MBs/GCE, c represents B-Cap/S-MBs/GCE, d represents BSA/B-Cap/S-MBs/GCE, e represents T/BSA/B-Cap/S-MBs/GCE, f represents Ab-5mC/T/BSA/B-Cap/S-MBs/GCE, and g represents Ab2-Ru@SiO₂/Ab-5mC/T/BSA/B-Cap/S-MBs/GCE.

For the bare electrode (a) of the magnetic glass carbon electrode or S-MB and different composites modified with S-MB and different composites (b-g), after the S-MB and composites were added to the GCE, FIG. 7 represents stepwise increase of the resistance of the electrode surface, decreased electron transfer efficiency, stepwise decrease of the corresponding redox current (FIG. 7A: a-g), as well as stepwise increase of the EIS impedance increased stepwise (FIG. 7B: a-g), due to the low conductivities of the magnetic beads and the composites, as recognized.

(2) Optimization of Electrochemical Parameters for ECL Sensor System in this Embodiment

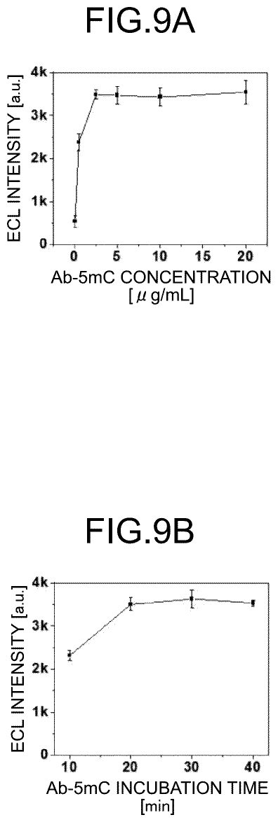

The pH value and TPrA concentration of the ECL detection electrolyte used in Example 3 were changed in the range of 6.0 to 8.5 for the pH and in the range of 10 mM to 80 mM for the TPrA concentration. The results are illustrated in FIGS. 8A and 8B. FIGS. 8A and 8B illustrate the ECL response status of Ru@SiO₂ prepared in Example 3 under different ECL detection conditions. FIG. 8A represents the relationship between different TPrA concentrations and ECL intensity. FIG. 8B represents the relationship between different pH of the ECL detection electrolyte and ECL intensity.

As recognized from FIG. 8, all of the ECL detection electrolytes each with a pH value of 6.5 or more exhibited good ECL intensity. A pH value of 7.4 is the most optimal pH in consideration of the highest ECL signal intensity. When the TPrA concentration is 10 mM or more, the electrolyte exhibited good results in all cases. 40 mM TPrA is optimal TPrA concentration in consideration of the highest ECL signal intensity.

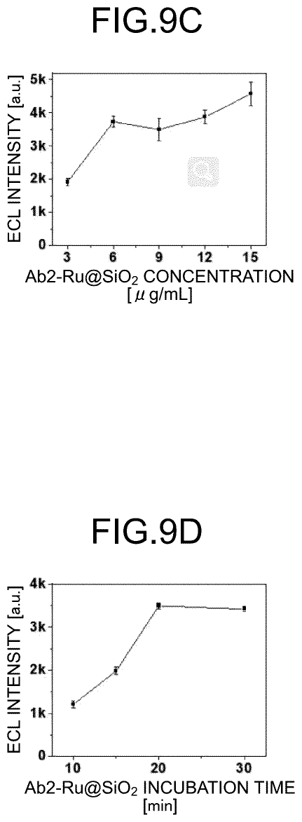

The Ab-5mC concentration, Ab-5mC incubation time, Ab2-Ru@SiO₂ concentration, and Ab2-Ru@SiO₂ incubation time in Example 3 were changed in the range of 0 to 20 μg/mL for the Ab-5mC concentration, in the range of 10 to 40 minutes for the Ab-5mC incubation time, in the range of 3 to 15 μg/mL for the Ab2-Ru@SiO₂ concentration, and in the range of 10 to 30 minutes for the Ab2-Ru@SiO₂ incubation time. FIGS. 9A to 9D represent the results. FIGS. 9A through 9D are diagrams representing the ECL response status of the ECL sensor system prepared in Example 3 under different detection conditions. FIG. 9A is a diagram representing the relationship between different Ab-5mC concentrations and ECL intensity. FIG. 9B is a diagram representing the relationship between different Ab-5mC incubation times and ECL intensity. FIG. 9C is a diagram representing the relationship between different Ab2-Ru@SiO₂ concentrations and ECL intensity. FIG. 9D is a diagram representing the relationship between different Ab2-Ru@SiO₂ incubation times and ECL intensity.

As recognized from FIGS. 9A through 9D, the Ab-5mC concentrations of 1 μg/mL or more achieved good ECL intensities in all cases. The Ab-5mC concentrations of 2.5 μg/mL or more achieved stable ECL signal intensity, and is thereby deemed optimal Ab-5mC concentrations. The Ab-5mC incubation time of 10 min or more achieved good ECL intensity in all cases. The Ab-5mC incubation time of 20 min or more achieved stable ECL signal intensity, and is thereby deemed optimal Ab-5mC incubation time. The Ab2-Ru@SiO₂ concentration of 4 μg/mL or more achieved good ECL intensity in all cases. The Ab2-Ru@SiO₂ concentration of 6 μg/mL or more achieved stable or higher ECL signal intensity, and is thereby deemed the optimal Ab2-Ru@SiO₂ concentration. The Ab2-Ru@SiO₂ incubation time of 15 min or more achieved good ECL intensity in all cases. The Ab2-Ru@SiO₂ incubation time of 20 min or more achieved stable ECL signal intensity, and is thereby deemed optimal Ab-5mC incubation time. It is preferable that a concentration of the electro-chemiluminescent nanoprobe is 4 μg/mL or more. It is preferable that an incubation time after addition of the electrochemiluminescent nanoprobe is 15 minutes or more.

### Example 5: Verification of the Feasibility of ECL Detection Method in this Embodiment

The composite was obtained by performing the steps in Example 3 described above, with use of T₀, T_{5mC} and background solution without T instead of T in Example 3

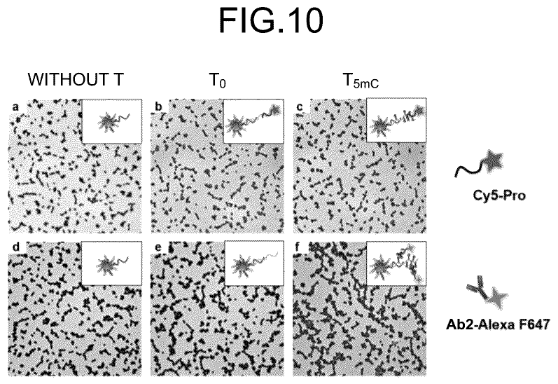

The composite was dispersed in PBS. Then, 3 μL was taken and placed on an ITO conductive glass, the excitation wavelength of 620 nm was applied to take the images with a fluorescence microscope (Leica DMi8 inverted microscope and Photometrics Prime 95B camera were disposed). The fluorescence micrographs obtained are illustrated in a, b and c in FIG. 10. FIG. 10 is a fluorescence verification diagram illustrating the principle of detection of the target nucleic acid by the ECL sensor system prepared in Example 3. In FIG. 10, symbols a, b, and c represent fluorescence imaging diagrams of blank solution (without T), negative control sequence (T₀), and target DNA sequence (T_{5mC}), respectively, when Cy5-Pro nucleic acid detection probe was used. Symbols d, e, and f represent fluorescence imaging diagrams of blank solution (without T), negative control sequence (T₀), and target DNA sequence (T_{5mC}), respectively, when fluorescence labeled secondary antibody (Ab2-Alexa F647) was used.

As recognized in a, b, and c in FIG. 10, S-MB/B-Cap without binding to the nucleic acid exhibited no fluorescence phenomenon. After bound to T₀ and T_{5mC}, the magnetic beads exhibited fluorescence. The results demonstrate that B-Cap can capture T₀ and T_{5mC}, and that the methylated sites do not affect the complementary pairing of the double-stranded bases, as well as that B-Cap can bind to S-MB.

Instead of Ab2 in Example 1, a fluorescence labeled secondary antibody (Ab2-Alexa F647) that can bind specifically to Ab-5mC was used to provide Ab2-Alexa F647-Ru@SiO₂ for use in Example 3. The composite obtained in Example 3 was photographed with a fluorescence microscope in the aforementioned method. Results are illustrated in d, e, and f in FIG. 10.

As illustrated in d, e, and f in FIG. 10, there was no fluorescence in any of the S-MB/B-Caps that were not bound to T and bound to T₀, while the magnetic beads bound to T_{5mC} exhibited fluorescence clearly. The results demonstrate that Ab-5mC can bind to methylation sites on the target DNA strand.

As recognized from the above results, this ECL detection method is a feasible method.

(3) Measurement of Detection Limit of ECL Sensor System in this Embodiment

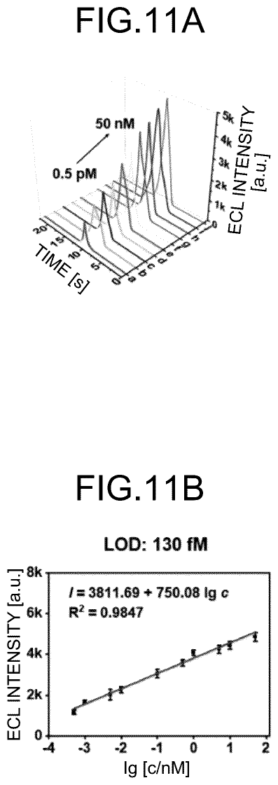

The concentration of methylated DNA in T in Example 3 was changed within the concentration range of 0.5 pM to 50,000 pM. The results are illustrated in FIG. 11A and FIG. 11B. FIGS. 11A and 11B illustrate the ECL response status at different concentrations of methylated DNA in the ECL sensor system prepared in Example 3. FIG. 11A represents the ECL response curves for different concentrations of methylated DNA, where a through i denote 0.5 pM, 1 pM, 10 pM, 50 pM, 100 pM, 500 pM, 10 nM and 50 nM, respectively. FIG. 11B represents a calibration curve for detection of the methylated DNA at different concentrations, n=3.

As recognized from FIGS. 11A and 11B, ECL intensity (I) increases with increase of methylated DNA concentration (FIG. 11A), and the logarithm of methylated DNA concentration and the ECL intensity (I) exhibited a linear relationship (FIG. 11B) with a correlation coefficient of 0.9915. The lowest limit of detection (LOD) for methylated DNA, calculated as the triple signal-to-noise (SN) ratio, was 0.13 pM.

(3) Verification of the Selectivity of ECL Detection Method in this Embodiment

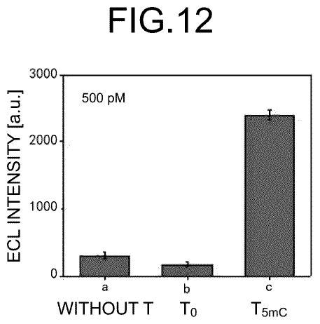

The ECL sensor system prepared in Example 3 was used to measure for T₀, T_{5mC}, and background solution. The results are illustrated in FIG. 12. FIG. 12 is a diagram representing the results of the evaluation of the selectivity of the ECL detection method in this embodiment.

As illustrated in FIG. 12, T₀ and background solution exhibited little ECL signal generated, while T_{5mC} exhibited high ECL intensity. This demonstrates superior selectivity of the ECL sensor system in this embodiment.

(4) Verification of Stability of ECL Detection Method in this Embodiment

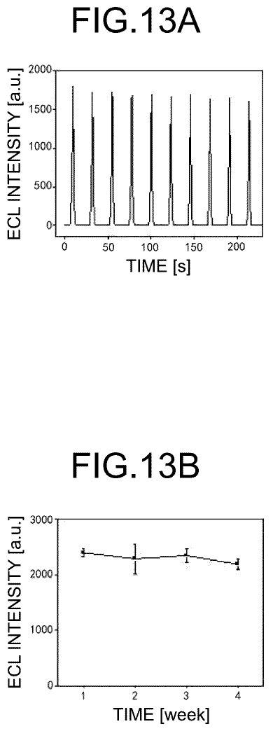

For the ECL sensor system in Example 3, a scanning potential of 0 V to +1.25 V was applied to the electrode containing T_{5mC} continuously 10 times within 0 to 250 seconds. The results are illustrated in FIG. 13A. FIGS. 13A and 13B are diagrams representing the results of the evaluation of the stability of the ECL detection method in this embodiment. FIG. 13A is a diagram representing the evaluation results of the signal stability of the ECL detection method in this embodiment. FIG. 13B is a diagram representing the evaluation results of the long-term detection stability of the ECL detection method in this embodiment.

FIG. 13A demonstrates stabilized ECL signal. This demonstrates superior stability of the ECL signal by the ECL detection method in this embodiment.

The reaction reagent 1 (B-Cap/S-MB) prepared was stored in PBS at 4° C. ECL detection was performed every 7 days on samples containing 500 pM of target DNA sequence (RASSF1A). The results are illustrated in FIG. 13B.

FIG. 13B demonstrates that the detection method in this embodiment has high detection stability within 28 days and superior long-term detection stability.

As revealed in the experimental results of the above Examples, the electrochemiluminescence detection method in this embodiment is simple and rapid, and achieves high detection efficiency at a low cost and feasibility for use in kit, as well as high detection sensitivity, the detection limit for the nucleic acid-specific site modification up to fM level.

The ECL detection method of this embodiment and the ECL nanoprobes used in the detection method and its preparation method, and the like, are described based on the embodiment. But, this embodiment is not limited to the above embodiment. This embodiment encompasses other embodiments obtained by applying various transformations that one skilled in the art can conceive of to the embodiment as well as other embodiments achieved by combining some components in the embodiment in the scope of this embodiment, as long as not departing from the scope of this embodiment.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A method for preparing an electrochemiluminescent nanoprobe, the method comprising:
adding a metal complex ion to an inorganic oxide nanoparticle to provide a metal-doped inorganic oxide nanoparticle; and
binding a secondary antibody to the metal-doped inorganic oxide nanoparticle to provide a metal-doped inorganic oxide nanoparticle modified with the secondary antibody, wherein
the secondary antibody is configured to identify a specific antibody against nucleic acid-specific site modification.

2. The preparation method according to claim 1, wherein the inorganic oxide nanoparticle is a silicon dioxide nanoparticle, a titanium dioxide nanoparticle, a zinc oxide nanoparticle or an iron oxide nanoparticle, or a nanoparticle coated with silicon dioxide, titanium dioxide, zinc oxide or iron oxide.

3. The preparation method according to claim 1, wherein the inorganic oxide nanoparticle is a silicon dioxide nanoparticle.

4. The preparation method according to claim 1, wherein the secondary antibody is a protein that identifies a generic portion of the specific antibody.

5. The preparation method according to claim 1, wherein the metal complex ion is tris(bipyridine)ruthenium(II) complex ion (Ru(bpy)₃²⁺).

6. An electrochemiluminescent nanoprobe prepared by a method of preparing an electrochemiluminescent nanoprobe, the method comprising:
adding a metal complex ion to an inorganic oxide nanoparticle to provide a metal-doped inorganic oxide nanoparticle; and
binding a secondary antibody to the metal-doped inorganic oxide nanoparticle to provide a metal-doped inorganic oxide nanoparticle modified with the secondary antibody, wherein
the secondary antibody is configured to identify a specific antibody against nucleic acid-specific site modification.

7. A method for electrochemiluminescence detection of nucleic acid-specific site modification, the method comprising:
a first step of mixing a sample to be detected with a first reaction reagent that is a capture nucleic acid modified magnetic bead to identify and capture a modification of a target nucleic acid in the sample;
a second step of capturing and labeling a specific site modification of the target nucleic acid, with use of a second reaction reagent that is a specific antibody against the nucleic acid specific site modification;
a third step of performing detection signal labeling to a composite of a magnetic bead capture nucleic acid-target nucleic acid-specific antibody provided at the second step with use of a third reaction reagent that is an electrochemiluminescent nanoprobe prepared by a method of preparing an electrochemiluminescent nanoprobe, the method comprising: adding a metal complex ion to an inorganic oxide nanoparticle to provide a metal-doped inorganic oxide nanoparticle; and binding a secondary antibody to the metal-doped inorganic oxide nanoparticle to provide a metal-doped inorganic oxide nanoparticle modified with the secondary antibody, wherein the secondary antibody is configured to identify a specific antibody against nucleic acid-specific site modification; and
a fourth step of placing the composite of the magnetic bead capture nucleic acid-target nucleic acid-specific antibody-nanoprobe obtained at the third step on an electrode surface, adding a co-reactive agent and then performing electrochemiluminescence detection, and performing qualitative and quantitative analysis for the modification of the target nucleic acid based on the presence/absence and an intensity of an electrochemiluminescence signal.

8. The detection method according to claim 7, wherein the nucleic acid is deoxyribonucleic acid (DNA) or ribonucleic acid (RNA).

9. The detection method according to claim 7, wherein the specific site modification is a methylation modification, a methylolization modification, or a pseudouridylation modification.

10. The detection method according to claim 7, wherein the capture nucleic acid has a terminal to which biotin is bound and is modified to the magnetic bead by a biotin-streptavidin reaction.

11. The detection method according to claim 7, wherein the co-reactive agent is tripropylamine.

12. The detection method according to claim 7, wherein the electrode is one selected from a glass carbon electrode, an indium tin oxide (ITO) electrode, and a screen printed electrode.

13. The detection method according to claim 7, wherein pH of an electrochemiluminescent electrolyte used at the fourth step is 6.5 or more.

14. The detection method according to claim 7, wherein a concentration of the co-reactive agent is 10 mM or more.

15. The detection method according to claim 7, wherein a concentration of the specific antibody is 1 μg/mL or more.

16. The detection method according to claim 7, wherein an incubation time after addition of the specific antibody is 10 min or more.

17. The detection method according to claim 7, wherein a concentration of the electrochemiluminescent nanoprobe is 4 μg/mL or more.

18. The detection method according to claim 7, wherein an incubation time after addition of the electrochemiluminescent nanoprobe is 15 minutes or more.

19. A kit for a method of electrochemiluminescence detection with an antibody, the kit comprising an electrochemiluminescent nanoprobe prepared by an electrochemiluminescent nanoprobe preparation method including: adding a metal complex ion to an inorganic oxide nanoparticle to provide a metal-doped inorganic oxide nanoparticle; and binding a secondary antibody to the metal-doped inorganic oxide nanoparticle to provide a metal-doped inorganic oxide nanoparticle modified with the secondary antibody, the secondary antibody being configured to identify a specific antibody against nucleic acid-specific site modification.

20. A nanoparticle for an electrochemiluminescent nanoprobe, comprising an inorganic oxide doped with a metal complex ion.

21. An electrochemiluminescent nanoprobe comprising:
an inorganic oxide nanoparticle doped with a metal complex ion; and
a secondary antibody bound to the inorganic oxide nanoparticle.
